SEQUENCE LISTING (8 sequences)

SEQ ID NO: 1 - RNA, 81 nt, Artificial Sequence
  Synthetic; Tiled iOligos oligonucleotides
guuuuagagcuagaaauagcaaguuaaaauaaggcuaguccguuaucaacuugaaaaaguggcaccgagucggugcuuuuu

SEQ ID NO: 2 - RNA, 20 nt, Artificial Sequence
  Synthetic; Full length iOligo oligonucleotide
aaagcaccgacucggugcca

SEQ ID NO: 3 - RNA, 16 nt, Artificial Sequence
  Synthetic; 5′ truncated iOligo oligonucleotide
caccgacucggugcca

SEQ ID NO: 4 - RNA, 15 nt, Artificial Sequence
  Synthetic; 3′ truncated iOligo oligonucleotide
aaagcaccgacucgu

SEQ ID NO: 5 - RNA, 20 nt, Artificial Sequence
  Synthetic; Antagonist oligonucleotide
guuuuagagcuagaaauagc

SEQ ID NO: 6 - RNA, 20 nt, Artificial Sequence
  Synthetic; Antagonist oligonucleotide
aaguuaaaauaaggcuaguc

SEQ ID NO: 7 - RNA, 20 nt, Artificial Sequence
  Synthetic; Antagonist oligonucleotide
cguuaucaacuugaaaaagu

SEQ ID NO: 8 - RNA, 20 nt, Artificial Sequence
  Synthetic; Antagonist oligonucleotide
ggcaccgagucggugcuuuu

(19) United States
(12) Patent Application Publication
(10) Pub. No.: US 2022/0259597 A1
(43) Pub. Date: Aug. 18, 2022

(54) OLIGONUCLEOTIDE ANTAGONISTS FOR RNA GUIDED GENOME EDITING

(71) Applicant: Georgia Tech Research Corporation, Atlanta, GA (US)

(72) Inventors: James Everett Dahlman, Atlanta, GA (US); Cory Dane Sago, Atlanta, GA (US)

(21) Appl. No.: 17/629,325

(22) PCT Filed: Jul. 24, 2020
(86) PCT No.: PCT/US2020/043517

§ 371 Date: Jan. 21, 2022

Related U.S. Application Data
(60) Provisional application No. 62/879,961, filed on Jul. 29, 2019.

(51) Int. Cl. C12N 15/113; A61K 9/51

(52) U.S. Cl. CPC C12N 15/113; A61K 9/5123; C12N 2320/32; C12N 2310/20; C12N 2320/31; C12N 2310/14

(57) ABSTRACT

Compositions and methods for inactivating RNA-guided genome editing systems in specific tissue, for example hepatocytes, are provided herein. In one embodiment, the compositions are small chemically modified oligonucleotides that can target and bind to guide RNA, thus eliminating the ability of guide RNA to interact with an endonuclease. The disclosed oligonucleotides are delivered in lipid nanoparticles formulated to target a specific tissue. Subsequently delivered RNA-guided genome editing systems will be inhibited in the specific tissue that received the oligonucleotides. The disclosed compositions and methods allow for reduced RNA-guided genome editing in hepatocytes.

### INCORPORATION BY REFERENCE TO ANY PRIORITY APPLICATIONS

This application claims the benefit of U.S. Provisional Application No. 62/879,961 filed Jul. 29, 2019 entitled, “OLIGONUCLEOTIDE ANTAGONISTS FOR RNA GUIDED GENOME. EDITING”, which is incorporated by reference in its entirety.

### STATEMENT REGARDING FEDERALLY SPONSORED RESEARCH

This subject matter described herein was made with government support under R01DE026941 and T32EB021962 awarded by the National Institutes of Health. The government has certain rights in the invention.

### REFERENCE TO SEQUENCE LISTING

The present application is being filed along with an Electronic Sequence Listing. The Electronic Sequence Listing is provided as a file entitled GUIDE007WOSEQLIST.txt, created and last modified on Jul. 15, 2020, which is 2,042 bytes in size. The information in the electronic format of the Electronic Sequence Listing is incorporated herein by reference in its entirety.

### TECHNICAL FIELD

The subject natter described herein is generally related to the field of gene editing platforms. More specifically, this invention is related to compositions and methods for controlling gene editing activity.

### BACKGROUND

CRISPR-based genome editing systems have therapeutic promise (Doudna, J. A., et al., Science, 346:1258096 (2014)). However, their clinical utility is limited by ineffective drug delivery. Non-viral CRISPR therapies in adult animals have been limited to local delivery (Lee, B., et al., Nature Biomedical Engineering, 2:497-507 (2018); Lee, K., et al., Nature Biomedical Engineering, 1:889-901 (2017); Gao, X., et al., Nature, 553: 217-221 (2018)), or if administered systemically, preferentially editing in hepatocytes (Miller, J. B., et al., Angew Chem. Int Ed Engl, 56:1059-1063 (2018); Jiang, C., et al., Cell Research, 27:440-443 (2017); Yin, H., et al., Nat Biotechnol, 35:1179-1187 (2017); Finn, J. D., et al., Cell Rep, 22:2227-2235 (2018)). Unwanted hepatocyte delivery extends beyond CRISPR; many nanoparticles preferentially target hepatocytes (Lorenzen, C., et al., J Control Release, 203:1-15 (2015)). Thus, a pragmatic way to enable systemic, programmable, cell type-specific gene editing outside hepatocytes would constitute an important step for CRISPR therapeutics and nanomedicine.

To achieve non-hepatocyte drug delivery, scientists focus on increasing delivery to the new cell type. This is achieved by varying nanoparticle size, charge, chemical structure, or by adding targeting ligands that bind receptors on target cells (Blanco, E., et al., Nat Biotechnol, 33:941-951 (2015)). Yet off-target hepatocyte delivery remains an unsolved problem, since the structure of hepatic sinusoids promotes unwanted nanoparticle accumulation (Tsai, K. M., et al., Nat Mater, 15: 1212-1221 (2016)). Thus, the current paradigm for systemic ‘non-liver’ Cas9 therapies, which requires a nanoparticle to (i) efficiently target a new cell type and (ii) avoid hepatocytes, may be difficult to achieve in the short term. There is a need for new strategies to reduce or eliminate off-target hepatocyte delivery of therapeutic agents.

Therefore, it is an object of the invention to provide compositions and methods for inactivating genome editing systems in specific tissue.

It is another object of the invention to provide compositions and methods for reducing off-target side effects from gene editing drugs.

### SUMMARY

Compositions and methods for inactivating RNA-guided genome editing systems in specific cells, tissues, or organs are provided herein. The compositions are useful for mitigating gene editing in unwanted cells, tissues, or organs particularly when the gene editing compositions are administered systemically. One embodiment provides a pharmaceutical composition including genome editing antagonist oligonucleotide having a nucleic acid sequence complementary to at least a portion of a tracrRNA sequence of an sgRNA, wherein the oligonucleotide blocks, inhibits or interferes with the interaction of the sgRNA and an RNA-guided DNA endonuclease.

Another embodiment provides a pharmaceutical composition including (i) nanoparticles including a genome editing antagonist oligonucleotide having a nucleic acid sequence complementary to at least a portion of a tracrRNA sequence of an sgRNA, wherein the oligonucleotide blocks, inhibits or interferes with the interaction of the sgRNA and an RNA-guided DNA endonuclease, (ii) nanoparticles including a nucleic acid encoding the RNA-guided DNA endonuclease, and (iii) nanoparticles including the sgRNA, wherein the sgRNA has a first nucleic acid sequence including a crRNA sequence having complementarity to a nucleic acid sequence encoding a target gene fused to a second nucleic acid sequence including the tracrRNA sequence.

The disclosed genome editing antagonist oligonucleotides can be chemically modified to increase stability, reduce immunogenicity, or increase affinity between the genome editing antagonist oligonucleotide and the guide RNA. Exemplary modifications include 2′O-Methyl ribose or phosphorothioate.

In one embodiment, the RNA guided DNA endonuclease is selected from the group consisting of Cas9, CasX, CasY, and Cas13, or Cpf1.

In one embodiment, the genome editing antagonist oligonucleotides are delivered in a nanoparticle, for example a lipid nanoparticle. In certain embodiments, the nanoparticles preferentially target hepatocytes.

In one embodiment, the genome editing antagonist oligonucleotide are delivered in a lipid nanoparticle having a formulation including C₁₄PEG₂₀₀₀, cholesterol, 1,2-distearoyl-sn-glycero-3-phosphocholine (DSPC), and the ionizable lipid cKK-E12. The lipid nanoparticle can have 30 mol % to about 80 mol % cKK-E12, about 5 mol % to about 55 mol % cholesterol, about 10 mol % to about 35 mol % phospholipid, and about 0 mol % to about 20 mol % PEG-lipid.

In another embodiment, the nanoparticles having a nucleic acid encoding an RNA guided DNA endonuclease and the nanoparticles having guide RNA are formulated to deliver nucleic acids to splenic endothelial cells or lung endothelial cells. Nanoparticle formulated to deliver cargo to splenic endothelial cells or lung endothelial cells have a formulation including 7C1:cholesterol:C₁₄-PEG₂₀₀₀:18:1 lyso PC at a molar ratio of 50:23.5:6.5:20 or 7C1:cholesterol:C₁₄-PEG₂₀₀₀:DOPE at a molar ratio of 60:10:25:5.

Also provided are methods of inhibiting RNA-guided gene editing in a subject in need. An exemplary method includes pre-treating the subject with an effective amount of a pharmaceutical composition including a genome editing antagonist oligonucleotide having a nucleic acid sequence complementary to at least a portion of a tracrRNA sequence of an sgRNA, wherein the oligonucleotide blocks, inhibits or interferes with the interaction of the sgRNA and an RNA-guided DNA endonuclease, and wherein the pharmaceutical composition is formulated to deliver to hepatocytes, and after a period of time systemically administering to the subject a RNA-guided genome editing system in an amount effective to perform genome editing in cells. In one embodiment, the genome editing antagonist oligonucleotide is delivered to hepatocytes and inhibits the activity of the RNA-guided genome editing system genome editing system in the liver.

Also disclosed is a method of treating a genetic disease or disorder in a subject in need thereof by pre-treating the subject with an effective amount of a pharmaceutical composition including a genome editing antagonist oligonucleotide having a nucleic acid sequence complementary to at least a portion of a tracrRNA sequence of an sgRNA, wherein the oligonucleotide blocks, inhibits or interferes with the interaction of the sgRNA and an RNA-guided DNA endonuclease, and wherein the pharmaceutical composition is formulated to deliver to hepatocytes, and after a period of time administering to the subject an RNA-guided genome editing system in an amount effective to perform RNA-guided genome editing in diseased cells, wherein the effective amount of the pharmaceutical composition including a genome editing antagonist oligonucleotide inhibits the activity of the RNA-guided genome editing system in hepatocytes and genome editing occurs in other cell types, including the diseased cell. The genome editing oligonucleotide antagonist is administered to the subject 1, 2, 3, 4, or 5 hours before the RNA-guided genome editing system.

### BRIEF DESCRIPTION OF THE DRAWINGS

FIG. 1A is an illustration showing the interaction between SpCas9 and sgRNA which then interacts with, and edits, DNA. FIG. 1B is an illustration showing the proposed mechanism by which iOligo functions. By interacting with the conserved region of the sgRNA, the iOligo prevents Cas9-mediated gene editing. FIG. 1C is a schematic showing multiple iOligos tiled in the conserved region of an sgRNA backbone (SEQ ID NO:1).

FIG. 2A is a bar graph showing indel percent in Cas9-expressing cells following treatment with iOligo A, B, C, or D, or a scrambled control. FIG. 2B is a bar graph showing indel percent in Cas9-expressing cells following treatment with iOligos A, B, C, or D at various concentrations (150 nm, 50 nm, or 16 nm). FIG. 2C shows the sequence of full length (SEQ ID NO: 5′ truncated (SEQ ID NO:3), and 3′ truncated (SEQ ID NO:4) iOligo oligonucleotides. FIG. 2D is bar graph showing indel percent in Cas9-expressing cells after treatment with full-length and truncated versions of iOligo-D.

FIG. 3A is a bar graph showing normalized indel inhibition of iOligos with multiple ribose and linkage chemical modification patterns. FIG. 3B is a bar graph showing normalized indel inhibition in cells treated with different doses of iOligo chemically modified with phosphorothiote linkages and either 0-Methyl or Methoxyethyl riboses. FIG. 3C is a bar graph showing normalized indel inhibition in normal cells after iOligo treatment and Cas9 mRNA sgRNA treatment.

FIG. 4A is schematic showing the workflow of experimental iOligo treatment. Briefly, mice that constitutively express SpCas9 were pre-treated with iOligos delivered by a hepatocyte-trophic LNP Two hours later the same LNP was used to deliver sgGFP. FIG. 4B is a schematic showing the administration dose of iOligos. FIG. 4C is a bar graph showing normalized GFP mean fluorescence intensity (MFI) in hepatocytes from mice pre-treated with iOligo and mice pre-treated with control oligo. FIG. 4D is a bar graph showing normalized indel percentage in hepatocytes from mice pre-treated with control oligo (scramble) or iOligo.

FIG. 5A is a schematic showing the workflow of hepatocyte-trophic iOligo treatment. Briefly, wild-type mice were pre-treated with iOligos delivered by a hepatocyte-trophic LNP. Two hours later, the same mice were treated with LNPs carrying Cas9 mRNA and sgICAM-2. FIG. 5B is a bar graph showing normalized indel percentage in hepatocytes from mice pre-treated with iOligo and control oligo (scramble). FIG. 5C is a bar graph showing normalized indel percentage in splenic ECs from mice pre-treated with iOligo and control oligo (scramble).

FIG. 6A is a schematic illustration showing the workflow for combination iOligo and siGFP treatment. Briefly, wild-type mice were pre-treated with a combination of iOligo and siGFP. Mice received 1 mg/kg siCtrl or siGFP delivered by a hepatocyte-trophic LNP, then 1.2 mg/kg iOligos delivered by a hepatocyte-trophic LNP. Two hours later, the same mice were treated with 3 mg/kg Cas9 mRNA and sgICAM-2 delivered by a hepatocyte- and splenic EC-trophic LNP. FIG. 6B is a bar graph showing normalized indel percentage in hepatocytes for experimental groups pre-treated with combinations of control and active iOligos and siRNAs.

FIG. 6C is a bar graph showing normalized indel percentage in hepatocytes for experimental groups pre-treated with combinations of control and active iOligos and siRNAs. FIG. 6D is a bar graph showing the ratio of indels at on-target (splenic ECs) and off-target (hepatocytes) cells normalized to experimental group receiving control pre-treatment.

### DETAILED DESCRIPTION

Some embodiments relate to a pharmaceutical composition comprising: a plurality of nanoparticles comprising an effective amount of a genome editing antagonist oligonucleotide having a nucleic acid sequence complementary to at least a portion of a tracrRNA sequence of an sgRNA, wherein the oligonucleotide blocks, inhibits and/or interferes with the interaction of the sgRNA and an RNA-guided DNA endonuclease. In some embodiments, the genome editing antagonist oligonucleotide hybridizes to at least a portion of the tracrRNA sequence of the sgRNA. In some embodiments, the genome editing antagonist oligonucleotide is chemically modified to increase stability, reduce immunogenicity, and/or increase affinity between the genome editing antagonist oligonucleotide and the sgRNA.

In some embodiments, the modification is 2′O-Methyl ribose, phosphorothioate, or both. In some embodiments, the nanoparticles preferentially target hepatocytes. In some embodiments, the nanoparticles are lipid nanoparticles. In some embodiments, the lipid nanoparticles comprise C₁₄PEG₂₀₀₀, cholesterol, 1,2-distearoyl-sn-glycero-3-phosphocholine (DSPC), and an ionizable lipid, wherein the ionizable lipid is cKK-E12. In some embodiments, the lipid nanoparticles comprises about 30 mol % to about 80 mol % cKK-E12, about 5 mol % to about 55 mol % cholesterol, about 10 mol % to about 35 mol % phospholipid, and about 0 mol % to about 20 mol % PEG-lipid. In some embodiments, the genome editing antagonist oligonucleotide has a nucleic acid sequence that is 80% or more homologous, 85% or more homologous, 90% or more homologous, 95% or more homologous, and/or 100% homologous to any one of SEQ ID NOs:5-8.

Some embodiments relate to pharmaceutical composition comprising: a plurality of first nanoparticles comprising a genome editing antagonist oligonucleotide having a first nucleic acid sequence complementary to at least a portion of a tracrRNA sequence of an sgRNA, wherein the oligonucleotide blocks, inhibits and/or interferes with the interaction of the sgRNA and an RNA-guided DNA endonuclease; a plurality of second nanoparticles comprising a second nucleic acid sequence encoding the RNA-guided DNA endonuclease; and a plurality of third nanoparticles comprising the sgRNA, wherein the sgRNA comprises a third nucleic acid sequence comprising a crRNA sequence having complementarity to a fourth nucleic acid sequence encoding a target gene fused to a fifth nucleic acid sequence comprising the tracrRNA sequence.

In some embodiments, the tracrRNA has a nucleic acid sequence that is 80% or more homologous, 85% or more homologous, 90% or more homologous, 95% or more homologous, and/or 100% homologous to SEQ ID NO: 1. In some embodiments, the genome editing antagonist oligonucleotide has a nucleic acid sequence is 80% or more homologous, 85% or more homologous, 90% or more homologous, 95% or more homologous, and/or 100% homologous to any one of SEQ ID NO:5-8. In some embodiments, the genome editing antagonist oligonucleotide is chemically modified to increase stability, reduce immunogenicity, and/or increase affinity between the genome editing antagonist oligonucleotide and the sgRNA.

In some embodiments, the modification is 2′O-Methyl ribose, phosphorothioate, or both. In some embodiments, the first nanoparticles passively target hepatocytes. In some embodiments, the nanoparticles are lipid nanoparticles. In some embodiments, the lipid nanoparticles comprise C₁₄PEG₂₀₀₀, cholesterol, 1,2-distearoyl-sn-glycero-3-phosphocholine (DSPC), and an ionizable lipid, wherein the ionizable lipid is cKK-E12. In some embodiments, the lipid nanoparticles comprise about 30 mol % to about 80 mol % cKK-E12, about 5 mol % to about 55 mol % cholesterol, about 10 mol % to about 35 mol % phospholipid, and about 0 mol % to about 20 mol % PEG-lipid.

In some embodiments, the RNA guided DNA endonuclease is selected from the group consisting of Cas9, CasX, CasY, Cas13, and Cpf1. In some embodiments, the second nanoparticles and the third nanoparticles are formulated to deliver nucleic acids to splenic endothelial cells and/or lung endothelial cells. In some embodiments, one or both of the second and third nanoparticles comprise 7C1:cholesterol:C₁₄-PEG₂₀₀₀:18:1 lyso PC at a molar ratio of 50:23.5:6.5:20 or 7C1:cholesterol:C₁₄-PEG₂₀₀₀:DOPE at a molar ratio of 60:10:25:5.

Some embodiments relate to a method of inhibiting RNA-guided gene editing in hepatocytes in a subject in need thereof comprising: pre-treating the subject with an effective amount of a pharmaceutical composition comprising a genome editing antagonist oligonucleotide having a nucleic acid sequence complementary to at least a portion of a tracrRNA sequence of an sgRNA, wherein the oligonucleotide blocks, inhibits and/or interferes with the interaction of the sgRNA and an RNA-guided DNA endonuclease, and wherein the pharmaceutical composition is formulated to deliver to hepatocytes, and after a period of time systemically administering to the subject a RNA-guided genome editing system in an amount effective to perform genome editing in cells, wherein the effective amount of the pharmaceutical composition inhibits the activity of the RNA-guided genome editing system in hepatocytes.

In some embodiments, the RNA-guided genome editing system comprises an RNA-guided endonuclease and an sgRNA. In some embodiments, the RNA-guided DNA endonuclease is Cas9. In some embodiments, the genome editing antagonist oligonucleotide is delivered in a nanoparticle. In some embodiments, the RNA-guided genome editing system is administered systemically.

Some embodiments relate to a method of treating a genetic disease or disorder in a subject in need thereof comprising, pre-treating the subject with an effective amount of a pharmaceutical composition comprising a genome editing antagonist oligonucleotide having a nucleic acid sequence complementary to at least a portion of a tracrRNA sequence of an sgRNA, wherein the oligonucleotide blocks, inhibits and/or interferes with the interaction of the sgRNA and an RNA-guided DNA endonuclease, and wherein the pharmaceutical composition is formulated to deliver to hepatocytes, and after a period of time administering to the subject an RNA-guided genome editing system in an amount effective to perform RNA-guided genome editing in diseased cells, wherein the effective amount of the pharmaceutical composition inhibits the activity of the RNA-guided genome editing system in hepatocytes and genome editing occurs in other cell types, including the diseased cells.

In some embodiments, the RNA-guided genome editing system is administered systemically. In some embodiments, the genome editing antagonist oligonucleotide is administered to the subject 1, 2, 3, 4, or 5 hours before the RNA-guided genome editing system.

Some embodiments relate to a kit comprising: a plurality of first nanoparticles comprising a genome editing antagonist oligonucleotide having a first nucleic acid sequence complementary to at least a portion of a tracrRNA sequence of an sgRNA, wherein the oligonucleotide blocks, inhibits and/or interferes with the interaction of the sgRNA and an RNA-guided DNA endonuclease; a plurality of second nanoparticles comprising a second nucleic acid sequence encoding the RNA-guided DNA endonuclease; and a plurality of third nanoparticles comprising the sgRNA, wherein the sgRNA comprises a third nucleic acid sequence comprising a crRNA sequence having complementarity to a fourth nucleic acid sequence encoding a target gene fused to a fifth nucleic acid sequence comprising the tracrRNA sequence.

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx, +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the nonlimiting group consisting of small interfering RNA (siRNA), microRNA (miRNA), Dicer-substrate RNA (dsRNA), small hairpin RNA (shRNA), mRNA, single-guide RNA (sgRNA), cas9 mRNA, and mixtures thereof.

The terms “polypeptide”, “peptide”, and “protein”, may be used interchangeably to refer a string of at least three amino acids linked together by peptide bonds. Peptide may refer to an individual peptide or a collection of peptides. Peptides can contain natural amino acids, non-natural amino acids (i.e., compounds that do not occur in nature but that can be incorporated into a polypeptide chain), and/or amino acid analogs. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. Modifications may include cyclization of the peptide, the incorporation of D-amino acids, etc.

“Oligonucleotide” refers to short nucleic acid molecules. Oligonucleotides are typically between about 13 to about 25 nucleotides and are designed to hybridize specifically to DNA or RNA sequences.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, “complementary nucleic acid” or “complementary DNA” refers to a strand of DNA or RNA that will pair with, or complement, a second strand of DNA or RNA.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by the same series in reverse and then by approximately 30 base pairs known as “spacer DNA”. The spacers are short segments of DNA that are often derived from a bacterial virus or other foreign genetic element and may serve as a ‘memory’ of past exposures to facilitate an adaptive defense against future invasions.

“CRISPR-associated nuclease” or “Cas” refers to an enzyme that cuts DNA at a specific location in the genome so that nucleotide bases can then be added or removed.

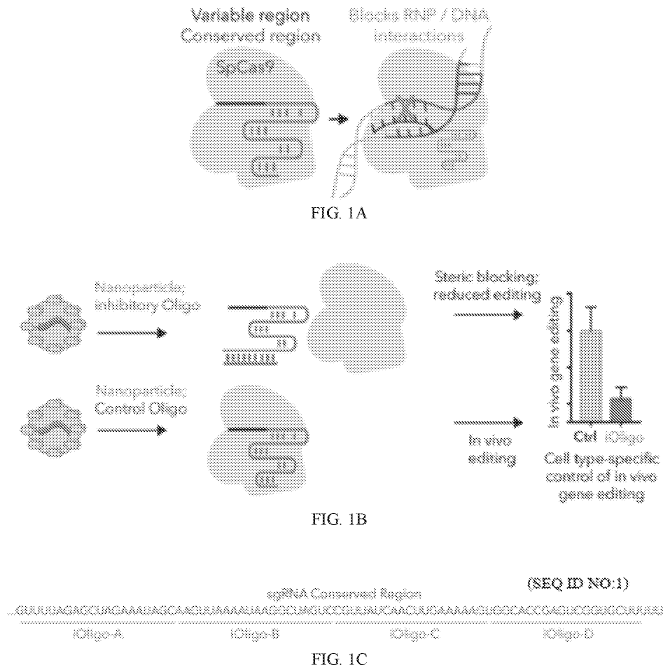

“Guide RNA” or “gRNA” refers to a specific RNA sequence that recognizes the target DNA region of interest and directs RNA-guided nucleases to the region of interest for editing. “Single guide RNA” or “sgRNA” refers to a single stranded guide RNA. The sgRNA includes two parts, crispr RNA (crRNA) and tracr RNA (as seen in FIG. 1A). crRNA is a 17-20 nucleotide sequence complementary to the target DNA which serves to direct Cas9 nuclease activity. tracr RNA serves as a binding scaffold for the Cas nuclease. Watson-Crick pairing of the sgRNA with the target site permits R-loop formation, which in conjunction with a functional PAM permits DNA cleavage or in the case of nuclease-deficient Cas9 allows tight binding to the DNA at that locus.

As used herein, “CRISPR genome editing system” refers to a guide RNA (gRNA or sgRNA) and a nuclease.

As used herein, the terms “treat,” “treating,” “treatment” and “therapeutic use” refer to the elimination, reduction or amelioration of one or more symptoms of a disease or disorder. As used herein, a “therapeutically effective amount” refers to that amount of a therapeutic agent sufficient to mediate a clinically relevant elimination, reduction or amelioration of such symptoms. An effect is clinically relevant if its magnitude is sufficient to impact the health or prognosis of a recipient subject. A therapeutically effective amount may refer to the amount of therapeutic agent sufficient to delay or minimize the onset of disease, e.g., delay or minimize the spread of cancer. A therapeutically effective amount may also refer to the amount of the therapeutic agent that provides a therapeutic benefit in the treatment or management of a disease.

As used herein, the term “prophylactic agent” refers to an agent that can be used in the prevention of a disorder or disease prior to the detection of any symptoms of such disorder or disease. A “prophylactically effective” amount is the amount of prophylactic agent sufficient to mediate such protection. A prophylactically effective amount may also refer to the amount of the prophylactic agent that provides a prophylactic benefit in the prevention of disease.

As used herein, the terms “individual,” “host,” “subject,” and “patient” are used interchangeably herein, and refer to a mammal, including, but not limited to, humans, rodents, such as mice and rats, and other laboratory animals.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

### II. Compositions for Regulating Genome Editing Platforms

Compositions and methods for inactivating RNA-guided genome editing systems in specific cells, tissues, or organs are provided herein. The disclosed genome editing antagonist compositions for inactivating, inhibiting, or reducing genome editing include, but are not limited to, small chemically modified oligonucleotides that can target and bind to guide RNA, thus eliminating the ability of guide RNA to interact with an engineered nuclease. In one embodiment, the guide RNA is single guide RNA (sgRNA) that includes a custom-designed targeting sequence (crRNA) fused to a scaffold tracrRNA sequence. In such an embodiment, the disclosed genome editing antagonist compositions hybridize to a portion of the tracrRNA sequence of the sgRNA.

When delivered systemically, RNA-guided genome editing systems preferentially perform genome editing in hepatocytes (Miller, J. B., et al., Angew Chem Int Ed Engl, 56:1059-1063 (2018); Jiang, C., et al., Cell Research, 27:440-443 (2017); Yin, H., et al., Nat Biotechnol, 35:1179-1187 (2017); Finn, J. D., et al., Cell Rep, 22:2227-2235 (2018)). In addition, hepatocyte delivery extends beyond CRISPR; many nanoparticles preferentially target hepatocytes (Lorenzer, C., et al., J Control Release, 203:1-15 (2015)). There is a need for more efficient tissue-specific RNA-guided genome editing, without unwanted genome editing in the liver. The disclosed genome editing antagonist compositions mitigate gene editing in unwanted cells, tissues, or organs particularly when the gene editing compositions are administered systemically.

The disclosed genome editing antagonist compositions offer many benefits over peptide- and protein-based genome editing antagonists. First, oligonucleotides are well tolerated in animals and humans (Adams, D., et al., N Engl J Med, 379:11-21 (2018)). Second, chemical modifications can increase oligonucleotide stability and potency (Deleavey, G. F., et al., Chem Biol, 19_937-954 (2012)). Third, lipid nanoparticles (LNPs) that deliver oligonucleotides to hepatocytes are clinically approved (Adams, D., et al., N Engl J Med, 379:11-21 (2018)), Finally, genome editing antagonist oligonucleotides can interact with the sgRNA and work independently of RNP complex formation (FIG. 1B).

Also disclosed is a drug delivery system that can be used to deliver genome editing antagonist compositions and genome editing system components to specific cells or tissues of interest. The disclosed approach can control the cell type-specific activity of genome editing drugs using genome editing antagonists. In one embodiment, the genome editing antagonist compositions can be delivered to a subject using nanoparticles.

A. Genome Editing Antagonist

One embodiment provides chemically modified genome editing antagonist oligonucleotides that inhibit or interfere with the interaction of guide RNA with engineered nucleases, effectively inhibiting genome editing. In one embodiment, the oligonucleotide targets a portion of a guide RNA that interacts with an engineered nuclease. The guide RNA can be single guide RNA (sgRNA) which is composed of a custom-designed targeting sequence (crRNA) fused to a trans-activating RNA (tracrRNA) sequence. The sgRNA directs Cas proteins to cleave any DNA containing a nucleotide target sequence complementary to the crRNA and adjacent PAM sequence. In one embodiment, the crRNA confers DNA target specificity, and the tracrRNA recruits the endonuclease to the sgRNA and the target nucleotide sequence. In one embodiment, the same tracrRNA sequence is used to create multiple sgRNAs, and the crRNA sequence is customized for each sequence that is to be targeted for genome editing.

Another embodiment provides genome editing antagonist oligonucleotides that hybridize to a portion of the tracrRNA sequence of an sgRNA. In one embodiment, the tracrRNA has a nucleic acid sequence according to SEQ ID NO: 1. Exemplary genome editing antagonist oligonucleotides are shown tiled across tracrRNA having a sequence according to SEQ ID NO:1 in FIG. 1C. The genome editing antagonist oligonucleotide can target any region of the tracrRNA, including the 5′ end and the 3′ end. In one embodiment, the genome editing antagonist oligonucleotide has a nucleic acid sequence according to any of the following:

(SEQ ID NO: 5)
GUUUUAGAGCUAGAAAUAGC
(SEQ ID NO: 6)
AAGUUAAAAUAAGGCUAGUC
(SEQ ID NO: 7)
CGUUAUCAACUUGAAAAAGU
(SEQ ID NO: 8)
GGCACCGAGUCGGUGCUUUU

In one embodiment, hybridization of the genome editing antagonist oligonucleotide to the tracrRNA inhibits, blocks, or interferes with the ability of tracrRNA to recruit the RNA-guided DNA endonuclease to the site of DNA cleavage. Without being bound by any one theory, it is believed that by blocking the ability of tracrRNA to recruit RNA-guided DNA endonucleases to the site of DNA cleavage, the RNA-guided DNA endonuclease will not be able to recognize and cleave the target gene sequence. In one embodiment, the disclosed genome editing antagonist oligonucleotides efficiently inhibit RNA-guided genome editing when used with an sgRNA engineered to contain a tracrRNA sequence complementary to the genome editing antagonist oligonucleotide.

The disclosed genome editing antagonist oligonucleotides can be used to regulate multiple sgRNAs simultaneously. In such an embodiment, the multiple sgRNAs are engineered to contain the same tracrRNA sequence but different crRNA sequences. The genome editing antagonist oligonucleotides are engineered to hybridize with the tracrRNA sequence common to all of the sgRNAs, and will therefore inhibit them regardless of their crRNA sequence. Therefore, one genome editing antagonist oligonucleotides can be used to regulate multiple sgRNAs.

The genome editing antagonist oligonucleotides can be modified to increase stability, reduce immunogenicity, and increase the affinity between the genome editing antagonist oligonucleotides and the tracrRNA. In one embodiment, the genome editing antagonist oligonucleotide has at least one chemically modified nucleotide. In some embodiments, the at least one chemically modified nucleotide comprises a chemically modified nucleobase, a chemically modified ribose, a chemically modified phosphodiester linkage, or a combination thereof. In some embodiments, the at least one chemically modified nucleotide is a chemically modified phosphodiester linkage. In some embodiments, the chemically modified phosphodiester linkage is phosphorothioate (PS). In one embodiment, the genome editing antagonist oligonucleotide is modified with 2′O-methyl ribose or phosphorothioate. Other exemplary modifications include but are not limited to 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl (2′-MOE), 2′-O-Allyl, 2′-O-Ethylamine, 2′-O-Cyanoethyl, (2′-Ome) Locked nucleic acid (LNA), Methylene-cLNA, N-MeO-amino BNA, or N-MeOaminooxy BNA.

In one embodiment, the genome editing antagonist oligonucleotide has at least 20 bases. In another embodiment, the genome editing antagonist oligonucleotide has between 14 bases and 20 bases. The genome editing antagonist oligonucleotide can have 12, 13, 14, 15, 16, 17, 18, 19, or 20 bases.

B. Delivery Vehicle

The disclosed genome editing antagonist oligonucleotides can be delivered to a cell or tissue by a delivery vehicle. In one embodiment, the delivery vehicle helps to carry the genome editing antagonist oligonucleotides to a specific cell type, for example hepatocytes, endothelial cells, or immune cells. The genome editing antagonist oligonucleotides can be passively delivered to hepatocytes in nanoparticles. In one embodiment, nanoparticles preferentially target hepatocytes. In one embodiment, the delivery vehicle is a nanoparticle composition.

In another embodiment, the genome editing antagonist oligonucleotides are delivered in a targeted delivery vehicle. The targeted delivery vehicle can be a lipid nanoparticle formulated to target a specific cell type.

1. Lipid Nanoparticle Delivery Vehicle

In one embodiment, the disclosed genome editing antagonist oligonucleotides are delivered to a site of interest in a lipid nanoparticle. The lipid nanoparticles can be formulated to target a specific cell type or tissue. In one embodiment, the lipid nanoparticle includes ionizable lipids, PEG lipids, phospholipids, and sterols. Exemplary lipid nanoparticle formulations are described in Dahlman, et al., Nat Nanotechnol 9:648-655 (2014), Yue, et al., PNAS, E3553-E3561 (2014), and Sago, et al., PNAS, 115: E9944-E9952 (2018).

a. Ionizable Lipids

In one embodiment, the disclosed lipid nanoparticles include an ionizable lipid. Ionizable lipids have a positive or partial positive charge at physiological pH. Exemplary ionizable lipids include but are not limited to 3,6-bis({4-[bis(2-hydroxydodecyl)amino]butyl}piperazine-2,5-dione (cKK-E12), 1-Linoleoyl-2-linoleyloxy-3-dmiethylaminopropane (DLin-2-DMAP), Dilinoleykarbanioyloxy-3-dimethylaniinopropane (DLin-C-DAP), 1,2-Dilmoleoyl-3-dimethylammopropane (DLm-DAP), 1,2-Dilinoleyloxy-N,N-dimethylaminopropane (DLin-DMA), 2,2-Dilinoleyl-4-dimethylaminomethy 1-[1,3]-dioxolane (DLin-K-DMA), 2,2-dilmoleyl-4-(2-dimethylaiiimoethyl)-[1,3]-dioxolane (DLin-KC2-DMA), (6Z,9Z,28Z,31Z)-heptatriaeonta-6,9,28,31-tetraen-19-yl 4-(dimethylamino)butanoate (DLin-MC3-DMA), 1,2-dioieoyl-3-dimethylammonium propane (DODAP), N,N-dimethyl-(2,3-dioleyloxy)propylamine (DODMA), dioctadecylamidoglycyocarboxysperrnine (DOGS), Spermine cholesterylcarbamate (GL-67), bis-guanidinium-spermidine-cholesterol (BGTC), 3β-(N— (N\hat{ }N′-dimethylammoethanej-carbamoxicholesterol (DC-Chol), N-t-butyl-N′-tetradecylamino-propionamidine (diC14-amidine), Dimethyldioctadecylammoniumbromide (DDAB), N-(1,2-dimyristyloxyprop-3-yl)-N,N-dimethyl-N-hydroxyethyl ammonium bromide (DMR1E), N,N-dioleyl-N,N-dimethylammonium chloride (DODAC), Dioleyloxypropyl-3-dimethyl hydroxyethyl ammonium bromide (DOME), N-(1-(2,3-dioleyloxy3)propyl)-N-2-(spenninecarboxamido)ethyl)-N,N-dimethylammonium trifluoracetate (DOSPA), 2-dioleoy trimethyl ammonium propane chloride (DOTAP), N-(1-(2,3-dioleyloxy)propyl)-N N,N-trimethylammonium chloride (DOTMA) Aminopropyl-dimethyl-bis(dodecyloxy)-propanaminiumbromide ((SAP-DLRIE), 1,2-dioleoyl-sn-3-phosphoethanolamine (“DOPE”), or combinations thereof. In a preferred embodiment, the ionizable lipid is cKK-E12. In one embodiment, the nanoparticle composition includes about 30 mol % to about 80 mol % ionizable lipid.

b. PEG-Lipids

The disclosed nanoparticle compositions also include one or more PEG or PEG-modified lipids. Such species may be alternately referred to as PEGylated lipids. Inclusion of a PEGylating lipid can be used to enhance lipid nanoparticle colloidal stability in vitro and circulation time in vivo. In some embodiments, the PEGylation is reversible in that the PEG moiety is gradually released in blood circulation. Exemplary PEG-lipids include but are not limited to PEG conjugated to saturated or unsaturated alkyl chains having a length of C3-C20. PEG-modified phosphatidylethanolamines, PEG-modified phosphatidic acids, PEG-modified ceramides (PEG-CER), PEG-modified dialkylamines, PEG-modified diacylglycerols (PEG-DAG), PEG-modified dialkylglycerols, and mixtures thereof. For example, a PEG lipid may be PEG-c-DOMG, PEG-DMG, PEG-DLPE, PEG-DMPE, PEG-DPPC or a PEG-DSPE lipid.

In one embodiment, the molecular weight of the PEG lipid can be modified to alter lipid nanoparticle tropism. The molecular weight of the PEG lipid can be 1 KDa, 2 KDa, or 3 KDa.

In a preferred embodiment, the PEG lipid is C₁₄PEG₂₀₀₀ or C₁₈PEG₂₀₀₀. In one embodiment, the nanoparticle composition includes about 0 mol % to about 20 mol % PEG lipid.

c. Phospholipids

The lipid component of a nanoparticle composition may include one or more phospholipids, such as one or more (poly)unsaturated lipids. Phospholipids may assemble into one or more lipid bilayers. In general, phospholipids may include a phospholipid moiety and one or more fatty acid moieties.

A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

Exemplary phospholipids include but are not limited to 1,2-distearoyl-sn-glycero-3-phosphocholine (DSPC), 1,2-dioleoyl-sn-glycero-3-phosphoethanolamine (DOPE), 1,2-dilinolenoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-0-octadecenyl-sn-glycero-3-phosphocholine (18:0 Diether PC), 1-oleoyl-2-cholesterylhemisuccinoy 1-sn-glycero-3-phosphocholine (OChemsPC), 1-hexadecyl-sn-glycero-3-phosphocholine (CI 6 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoyl phosphatidylglycerol (DPPG), palmitoyloleoylphosphatidylethanolam the (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidy ethanolamine (SOPS), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE). In a preferred embodiment, the phospholipid is DOPE. In one embodiment, the nanoparticle composition includes about 10 mol % to about 35 mol %.

d. Cargo

In one embodiment, the disclosed genome editing antagonist oligonucleotides are encapsulated within the lipid nanoparticle. In one embodiment, the lipid nanoparticle is dosed at less than 1.0 mg/kg genome editing antagonist oligonucleotides. The nanoparticle can contain 1.0, 0.9, 0.8, 0.7, 0.6, or 0.5 mg/kg genome editing antagonist oligonucleotides. In another embodiment, the lipid nanoparticle contains 0.5 mg/ml genome editing antagonist oligonucleotides.

In another embodiment, the disclosed genome editing antagonist oligonucleotides are part of a drug delivery system. In such an embodiment, the lipid nanoparticle compositions containing the disclosed genome editing antagonist oligonucleotides are formulated to deliver the oligonucleotides to a specific tissue before a second lipid nanoparticle composition delivers cargo to a second tissue or systemically. In one embodiment, the cargo encapsulated in the second lipid nanoparticle contains the components required for RNA-guided genome editing. The RNA-guided genome editing can be CRISPR/Cas based editing. CRISPR (Clustered Regularly Interspaced Short Palindromic Repeats) based gene editing requires two components: a guide-RNA and a CRISPR-associated endonuclease protein (Cas). In one embodiment, the second lipid nanoparticle composition includes sgRNA and a nucleic acid that encodes an RNA-guided endonuclease. Exemplary RNA-guided endonucleases include but are not limited to Cas9, CasX, CasY, Cas13, or Cpf1.

In one embodiment, the disclosed genome editing antagonist oligonucleotides can be combined with other gene editing methods. For example, lipid nanoparticle compositions containing the disclosed genome editing antagonist oligonucleotides can be delivered with lipid nanoparticles having siRNA cargo. Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer (Elbashir, et al. (2001) Nature, 411:494 498); (Ui-Tei, et al. (2000) FEBS Lett 479:79-82.) In one embodiment, the siRNA targets RNA-guided endonuclease snRNA. Exemplary RNA-guided endonucleases that can be targeted include but are not limited to Cas9, CasX, CasY, Cas13, or Cpf1. In one embodiment, the siRNA is delivered to the same target tissue as the genome editing antagonist oligonucleotides.

e. Exemplary Tissue Specific Lipid Nanoparticle Formulations

In one embodiment, the lipid nanoparticle carrying the disclosed genome editing antagonist oligonucleotides will target a specific cell-type, for example hepatocytes. An exemplary formulation for a hepatocyte targeting lipid nanoparticle includes C₁₄PEG₂₀₀₀, cholesterol, 1,2-distearoyl-sn-glycero-3-phosphocholine (DSPC), and the ionizable lipid cKK-E12. The lipid nanoparticle can include 30 mol % to about 80 mol % ionizable lipid, about 5 mol % to about 55 mol % cholesterol, about 10 mol % to about 35 mol % phospholipid, and about 0 mol % to about 20 mol % PEG-lipid.

In another embodiment, the lipid nanoparticle compositions containing the disclosed genome editing antagonist oligonucleotides are delivered to hepatocytes in a subject before a second lipid nanoparticle composition containing a gene editing platform are delivered systemically or to another tissue in the subject, such as lung or spleen endothelial cells. Therefore, lipid nanoparticle formulations targeting lung and spleen endothelial cells are also disclosed herein. 7C1 is a compound that has been shown to create lipid nanoparticles that can deliver materials to endothelial cells, 7C1 is synthesized by reacting C₁₅ epoxide-terminated lipids with PEI₆₀₀ at a 14:1 molar ratio (Dahlman, J., et al., Nat Nanotechnol, 9(8):648-655 (2014)). 7C1 has a structure according to Formula 1:

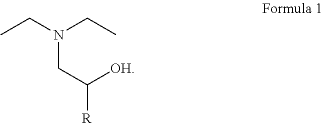

In one embodiment, exemplary lipid nanoparticle compositions to deliver sgRNA and Cas9 to lung endothelial cells include 7C1, cholesterol, C₁₄-PEG₂₀₀₀, and 18:1 lyso PC at a molar ratio of 50:23.5:6.5:20. In another embodiment, lipid nanoparticle compositions to deliver sgRNA and Cas9 to spleen endothelial cells include 7C1, cholesterol, C₁₄-PEG₂₀₀₀, and DOPE at a molar ratio of 60:10:25:5.

In another embodiment, the lipid nanoparticle compositions containing the disclosed genome editing antagonist oligonucleotides are delivered to immune cells in a subject before a second lipid nanoparticle composition containing a gene editing system are delivered systemically or to another targeted tissue in the subject. Therefore, lipid nanoparticle formulations targeting immune cells are also disclosed herein. It has been discovered that lipid nanoparticles having constrained lipids can more effectively deliver nucleic acids to specific tissues in the body, such as T cells. In one embodiment, lipid nanoparticles can be formulated by mixing nucleic acids with conformationally constrained ionizable lipids. PEG-lipids, phospholipids, cholesterol, and optionally a nucleic acid. An exemplary lipid nanoparticle formulation includes the conformationally constrained ionizable lipid 3-[(1-Adamantanyl)acetoxy]-2-{[3-(diethylamino)propoxycarbonyloxy]methyl}propyl (9Z,12Z)-9,12-octadecadienoate, a PEG-lipid, 1,2-Distearoyl-sn-glycero-3-phosphocholine (DSPC), and cholesterol. In one embodiment, the lipid nanoparticle formulation includes about 30 mol % to about 70 mol % conformationally constrained ionizable lipid, about 5 mol % to about 25 mol % phospholipid, about 25 mol % to about 45 mol % cholesterol, and about 0 mol % to about 5 mol % PEG-lipid. In another embodiment, the lipid nanoparticle formulation include about 35 mol % conformationally constrained ionizable lipid, about 16 mol % phospholipid, about 46.5 mol % cholesterol, and about 2.5 mol PEG-lipid.

In another embodiment, the antagonist oligonucleotide is attached to a targeting ligand conjugate and is not formulated in any transfection agent.

C. Pharmaceutical Compositions

Pharmaceutical compositions containing the disclosed genome editing antagonist oligonucleotides are provided herein. In one embodiment, the genome editing antagonist oligonucleotides are containing in nanoparticles. Nanoparticle compositions may be formulated in whole or in part as pharmaceutical compositions. Pharmaceutical compositions may include one or more nanoparticle compositions. For example, a pharmaceutical composition may include one or more nanoparticle compositions including one or more different therapeutic and/or prophylactics. Pharmaceutical compositions may further include one or more pharmaceutically acceptable excipients or accessory ingredients such as those described herein.

The lipid nanoparticle formulations targeting different cell-types can be administered together or separately. For examples, a pharmaceutical composition including hepatocyte targeting lipid nanoparticles can be administered to a subject before a pharmaceutical composition including endothelial cell targeting lipid nanoparticles. Alternatively, the hepatocyte targeting lipid nanoparticles and the endothelial cell targeting lipid nanoparticles can be delivered in the same pharmaceutical composition.

Pharmaceutical compositions including the disclosed genome editing antagonist oligonucleotides are provided. Pharmaceutical compositions containing the genome editing antagonist oligonucleotides can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed genome editing antagonist oligonucleotides, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For the disclosed genome editing antagonist oligonucleotides, generally dosage levels of 0.01 to 5 mg/kg of body weight daily are administered to mammals. More specifically, a preferential dose for the disclosed nanoparticles is 0.05 to 0.25 mg/kg. Generally, for intravenous injection or infusion, dosage may be lower.

In certain embodiments, the genome editing antagonist oligonucleotide composition is administered locally, for example by injection directly into a site to be treated. Typically, the injection causes an increased localized concentration of the genome editing antagonist oligonucleotide composition which is greater than that which can be achieved by systemic administration. The genome editing antagonist oligonucleotide compositions can be combined with a matrix as described above to assist in creating an increased localized concentration of the polypeptide compositions by reducing the passive diffusion of the polypeptides out of the site to be treated.

1. Formulations for Parenteral Administration

In some embodiments, compositions disclosed herein, including those containing genome editing antagonist oligonucleotides, are administered in an aqueous solution, by parenteral injection. The formulation may also be in the form of a suspension or emulsion. In general, pharmaceutical compositions are provided including effective amounts of a genome editing antagonist oligonucleotides, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Controlled Delivery Polymeric Matrices

The genome editing antagonist oligonucleotides disclosed herein can also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where the agent is dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of lipid nanoparticles, although in some embodiments biodegradable matrices are preferred. These may be natural or synthetic polymers, although synthetic polymers are preferred in some embodiments due to the better characterization of degradation and release profiles. The polymer is selected based on the period over which release is desired. In some cases, linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### III. Methods of Manufacturing Lipid Nanoparticles

Methods of manufacturing lipid nanoparticles are known in the art. In one embodiment, the disclosed lipid nanoparticles are manufactured using microfluidics. For exemplary methods of using microfluidics to form lipid nanoparticles, see Leung, A. K. K, et al., J Phys Chem, 116:18440-184:50 (2012), Chen. D., et al., J Am Chem &c, 134:6947-6951 (2012), and Belliveau, N. M., et al., Molecular Therapy-Nucleic Acids, 1: e37 (2012). Briefly, the cargo, such as an oligonucleotide or siRNA, is prepared in one buffer. The other lipid nanoparticle components (for example, ionizable lipid, PEG-lipid, cholesterol, and DOPE/DSPC) are prepared in another buffer. A syringe pump introduces the two solutions into a microfluidic device. The two solutions come into contact within the microfluidic device to form lipid nanoparticles encapsulating the cargo.

Methods of screening the disclosed lipid nanoparticles are discussed in International Patent Application No. PCT/US/2018/058171, which is incorporated by reference in its entirety. The screening methods characterizes vehicle delivery formulations to identify formulations with a desired tropism and that deliver functional cargo to the cytoplasm of specific cells. The screening method uses a reporter that has a functionality that can be detected when delivered to the cell. Detecting the function of the reporter in the cell indicates that the formulation of the delivery vehicle will deliver functional cargo to the cell. A chemical composition identifier is included in each different delivery vehicle formulation to keep track of the chemical composition specific for each different delivery vehicle formulation. In one embodiment, the chemical composition identifier is a nucleic acid barcode. The sequence of the nucleic acid bar code is paired to the chemical components used to formulate the delivery vehicle in which it is loaded so that when the nucleic acid bar code is sequenced, the chemical composition of the delivery vehicle that delivered the barcode is identified. Representative reporters include, but are not limited to siRNA, mRNA, nuclease protein, nuclease mRNA, small molecules, epigenetic modifiers, and phenotypic modifiers.

### IV. Methods of Use

A. Controlling Gene Editing

The disclosed genome editing antagonist oligonucleotides can be used to control the activity of RNA-guided genome editing platforms. Systemically delivered genome editing platforms have ineffective drug delivery and tend to preferentially target and perform gene editing in hepatocytes, leading to side effects and toxicity. In one embodiment, the genome editing antagonists inhibit RNA-guided genome editing in the liver, for example in hepatocytes. The genome editing antagonists can be used to reduce unwanted genome editing in specific tissues. Exemplary gene editing platforms include but are not limited to engineered nuclease editing systems such as CRISPR/Cas, zinc finger nucleases (ZEN), and transcription activator-like effector nucleases (TALEN). In one embodiment, the disclosed genome editing antagonist oligonucleotides are delivered to a subject systemically in a nanoparticle formulation. The nanoparticle formulation can preferentially target a specific cell type, tissue, or organ. In one embodiment, the nanoparticle formulation preferentially targets the liver.

In one embodiment, the gene editing platform is CRISPR/Cas. In such an embodiment, the subject is pre-treated with a pharmaceutical composition including at least one of the disclosed genome editing antagonist oligonucleotides. The genome editing antagonist oligonucleotides are delivered to hepatocytes via passive or targeted delivery vehicles. After a period of time has passed, the subject is administered a pharmaceutical composition including an RNA-guided genome editing system. In one embodiment, the RNA-guided genome editing system includes at least one sgRNA, and at least one nucleic acid encoding at least one Cas nuclease. In such an embodiment, the sgRNA includes a crRNA sequence complementary to a nucleic acid sequence in a target gene, and a tracrRNA sequence with complementarity to the genome editing antagonist oligonucleotides pre-delivered to the subject. The RNA-guided genome editing system can be delivered systemically or to a specific tissue. In one embodiment, the RNA-guided genome editing system and the and genome editing antagonist oligonucleotides are delivered to the same target cell type. The genome editing antagonist oligonucleotides inhibit the activity of the RNA-guided genome editing system only in tissues in which both components are present. For example, if the genome editing antagonist oligonucleotides are delivered to the liver, and the RNA-guided genome editing system is delivered systemically, the RNA-guided genome editing system will perform gene editing in all tissues that it reaches, with the exception of the liver. The genome editing antagonist oligonucleotides that were delivered to the liver will inhibit the action of the RNA-guided genome editing system by hybridizing to the tracrRNA sequence of the sgRNA and blocking its ability to recruit or interact with Cas.

In one embodiment, the first pharmaceutical composition including the genome editing antagonist oligonucleotides is administered to the subject 1, 2, 3, 4, 5, 6, 7, 8, or more than 8 hours before the second pharmaceutical composition including a CRISPR genome editing system. In another embodiment, the first pharmaceutical composition including the disclosed genome editing antagonist oligonucleotides is administered to the subject at least 1, 2, 3, 4, 5, or 7 days prior to administration of the second pharmaceutical composition including a CRISPR genome editing system.

In another embodiment, the disclosed genome editing antagonist oligonucleotides can be used with multiple sgRNAs simultaneously. In such an embodiment, the multiple sgRNAs are engineered to contain the same tracrRNA sequence but different crRNA sequences, thus targeting potentially different genes or different segments of a gene. The genome editing antagonist oligonucleotides are engineered to hybridize with the tracrRNA sequence common to all of the sgRNAs, and will therefore inhibit them regardless of their crRNA sequence. Therefore, one genome editing antagonist oligonucleotide can be delivered to the liver and will inhibit any sgRNAs that reach the liver and contain a tracrRNA sequence complementary to the genome editing antagonist oligonucleotide.

B. Diseases to be Treated

In one embodiment, the disclosed genome editing antagonist oligonucleotides are used to treat or reduce genetic diseases. An exemplary method includes pretreating a subject with at least one of the disclosed genome editing antagonist oligonucleotides targeted to the liver, and, after a period of time, systemically administering a gene editing system to the subject in an amount effective to promote genome editing in the subject in need thereof. In one embodiment, the subject is pre-treated with the genome editing antagonist oligonucleotides at least 1, 2, 3, 4, 5, 6, 7, or 8 hours before the genome editing system. In another embodiment, the genome editing antagonist oligonucleotides are administered to the subject 1, 2, 3, 4, or 5 days before the genome editing system.

Gene editing platforms can be applied to many genetic diseases and disorders. Exemplary genetic diseases and disorders associated with gene mutations include but are not limited to Alzheimer's disease, Angelman syndrome, Canavan disease, Charcot-Marie-Tooth disease, color blindness, Cri du chat, Crohn's disease, cystic fibrosis, down syndrome, Duchenne muscular dystrophy, familial hypercholesterolemia, haemochromatosis, hemophilia, Klinefelter syndrome, Lynch syndrome, muscular dystrophy, neurofibromatosis, phenylketonuria, polycystic kidney disease, Prader-Willi syndrome, Sickle cell disease, spinal muscular atrophy, Tay-Sachs disease, and Turner syndrome. In one embodiment, the genome editing system excises the mutated gene from the subject's genome or repairs the mutated gene to treat or reduce symptoms of the genetic disease or disorder in affected cells without performing genome editing in off target cells or tissues.

In another embodiment, genome editing can be used to treat or reduce cancer. Exemplary gene mutations associated with cancer include but are not limited to mutations in ABL, APC, AKT, ATN, AXIN, BCL-2, BRAF, BRCA1/2, CASP8, CCND1, CDKN1B, CDKN2A, CTNNB1, DNMT1, DINMT3A, EGFR, ERBB2, ERK, FGFR3, FLT3, GATA1/2/3, HERZ HRAS, JAK1/2/3, KIT, KLF4, KRAS, MAP2K1, MAP3K1, MET, MSH2, MYC, MYD88, NOTCH1/2, NRAS, p53, PIK3, PTEN, RAS, RAF, RBI, RET, SMAD2/4, SOX9, STAG2, STAT, STK11, TET2, TGF-β, TP53, TRAF7, VHL, and WT1. In one embodiment, the genome editing system excises the mutated gene from the cancer cell genome without performing gene editing in off-target cells.

C. Non-Clinical Applications

In another embodiment, the disclosed genome editing antagonist oligonucleotides can be used in a laboratory research setting. Genome editing can be used to generate animal models of disease. CRISPR-Cas systems can be used to rapidly and efficiently engineer one or multiple genetic changes to murine embryonic stem cells for the generation of genetically modified mice. In one embodiment, the disclosed genome editing antagonist oligonucleotides can be used to control cell-specific knockout of genes in laboratory animals, such as but not limited to mice, rats, primates, zebrafish, chickens, goats, cows, pigs, and dogs.

In another embodiment, the disclosed genome editing antagonist oligonucleotides can be used to control gene editing in plants to characterize gene functions and improve agricultural traits. The disclosed compositions and methods can be used to modify plants for the following non-limiting examples such as improving crop yield, improving nutritional profiles of crops, improving shelf life of fruits and vegetables, creating herbicide-resistant crops, and adapting plants to harsh environments in which they would not naturally grow, for example cold or arid regions.

### IV. Kits

Medical kits are also disclosed. The medical kits can include, for example, a dosage supply of one or more of the genome editing antagonist oligonucleotide disclosed herein. The genome editing antagonist oligonucleotide(s) can be supplied alone (e.g., lyophilized), in a pharmaceutical composition, or in a lipid nanoparticle formulation. The genome editing antagonist oligonucleotide(s) can be in a unit dosage, or in a stock that should be diluted prior to administration. In some embodiments, the kit includes a supply of pharmaceutically acceptable carrier. The kit can also include devices for administration of the active agent(s) or composition(s), for example, syringes. The kits can include printed instructions for administering the compound in a use as described above.

Kits designed for the above-described methods are also provided. In one embodiment the kits can include the disclosed lipid nanoparticles containing genome editing antagonist oligonucleotide, lipid nanoparticles containing a nucleic acid encoding an RNA-guided DNA endonuclease, and nanoparticles containing an sgRNA.

### EXAMPLES

### Example 1. Small Genome Editing Antagonist Oligonucleotide Called Inhibitory Oligos (iOligos Inhibit Cas9 Activity In Vitro

### Materials and Methods:

iOligo sequences were tiled across the conserved region of sgRNA (Jinek, M., et al., Science, 337:816-821 (2012)) (FIG. 1C). Each iOligo was chemically modified at every position with 2′O-methyl ribose and phosphorothioate modifications to increase stability, reduce immunogenicity, and increase affinity between the iOligo and target RNA (25268896). Initial experiments were performed in immortalized aortic endothelial cells (iMAECs) (Ni, C. W., et al., Vascular Cell, 6:7 (2014)) which were transduced with lentivirus to stably express SpCas9 (hereafter termed Cas9-iMAECs). Using Lipofectamine 2000, iOligos were transfected into Cas9-iMAECs. Four hours later, the same cells were transfected with 16 nM sgRNA targeting ICAM-2 (sgICAM-2). Seventy-two hours later, genomic DNA was isolated from the cells and insertions and deletions (indels) were quantified using Tracking of Indels by Decomposition (TIDE) (Brinkman, E. K., et al., Nucleic Acids Res, 42: e168 (2014)).

### Results:

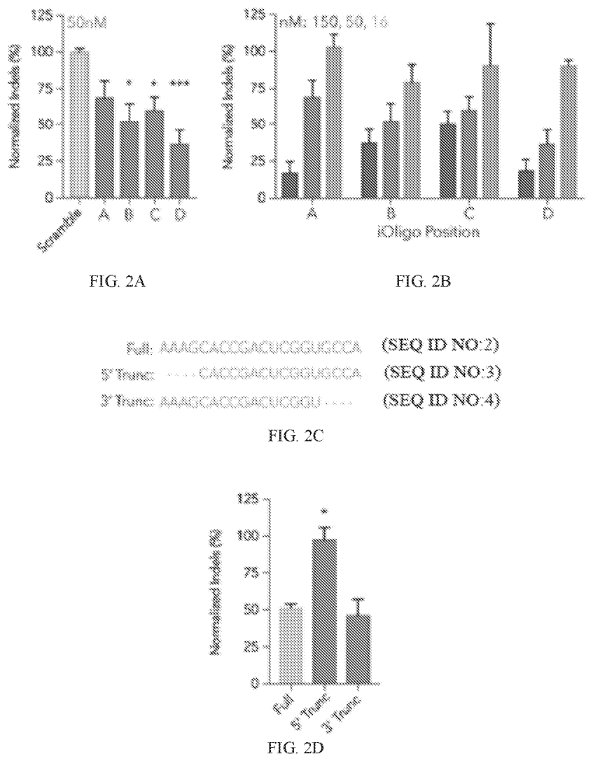

The ability of small chemically modified oligonucleotides to act as a universal genome editing antagonist to Cas9 was investigated. Compared to a scrambled oligonucleotide (same length, with the same chemical modifications), which acted as a control, all four iOligos reduced Cas9-mediated indels, suggesting the iOligos can block sgRNA activity in murine iOligo-D, which was targeted to the 3′ end of the sgRNA, reduced indels more than other iOligos (FIG. 2A). All four iOligos reduced indel formation in a dose-dependent way in Cas9-iMAECs (FIG. 2B) and exhibited ED50 values between 53 and 91 nM (Table 1). iOligo-D (hereafter termed iOligo) was selected for further studies.

TABLE 1
Calculated Effective Dose of Each iOligo

| Position | ED₅₀ (nM) |
|---|---|
| A | 67.8 |
| B | 70 |
| C | 91.6 |
| D | 53.2 |

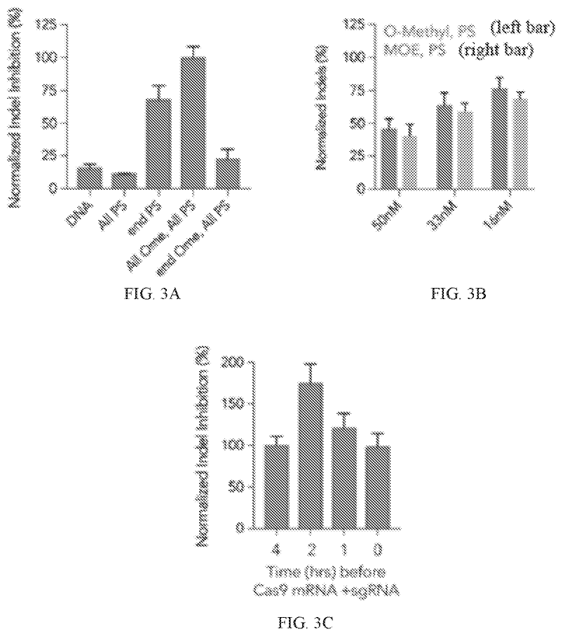

To probe the relationship between iOligo structure and anti-sgRNA activity, iOligo mutants were created by truncating four nucleotides from the 5′ and 3′, respectively. When the iOligo mutants were administered to Cas9-iMAECs at a 50 nM dose; the 5′ truncated mutant lost activity, since it did not block Cas9 gene editing. The 3′ truncated mutant maintained as much activity as the non-mutant iOligo, suggesting that iOligo potency depends on the sgRNA region that was targeted, more than iOligo length (FIG. 2D). To study the relationship between iOligo chemical modifications and anti-sgRNA activity, 50 nM iOligos with fewer modifications were administered to Cas9-iMAECs. The ‘original’ iOligo (i.e., fully modified) outperformed all iOligo variants with fewer modifications (FIG. 3A). These results also led to the conclusion that iOligos are unlikely to act via RNase H-mediated degradation of sgRNA, since fully 2′ O-methyl modifications prevent DNase H activity. As further evidence for this DNAse H-independent mechanism, 2′ Methoxyethyl modifications did not increase the efficacy of iOligo compared to 2′ O-methyl modifications (FIG. 313).

To confirm these results, which were all generated in Cas9 expression iMAECs, the ability of iOligos to maintain functionality when Cas9 was delivered transiently via mRNA was investigated. iOligos were transfected at a dose of 16 nM, then normal iMAECs (i.e., iMAECs that were not transduced with lentiviral-Cas9) were transfected with 300 ng Cas9 mRNA and 16 nM sgICAM-2. As expected, iOligos reduced indel formation. The time between iOligo administration and Cas9 administration was then varied iOligo efficacy was most effective 2 hours prior to the delivery of mRNA and sgRNA (FIG. 3C). Taken together, these results led to the conclusion that chemically modified, small oligonucleotides can block Cas9 activity in vitro.

### Example 2. iOligos can Control Systemic Gene Editing Therapies In Vivo

### Materials and Methods:

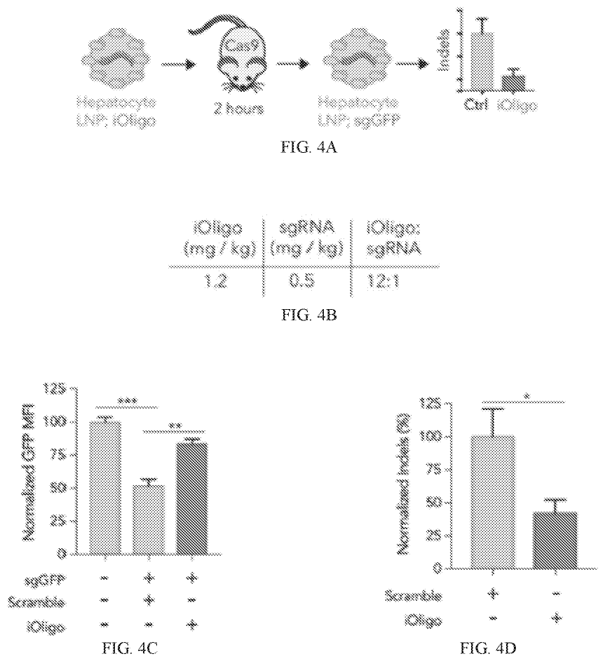

Anti-CRISPR studies have been performed in biochemical assays and cell culture (Pawluk, A., et al., Cell, 167:1829-1838 (2016); Shin, 1, et al., Science Advances, 3:e1701620 (2017); Zhu, Y., et al., BMC Biology, 16:32 (2018)), Thus, the ability of iOligo to control gene editing in adult mice using several models was investigated. First, gene editing was reduced in hepatocytes (FIG. 4A). Hepatocyte-targeting LNPs were formulated by mixing C14PEG₂₀₀₀, cholesterol, 1,2-distearoyl-sn-glycero-3-phosphocholine (DSPC), and the ionizable lipid cKK-E12. (Dong, Y., et al., PNAS, 111:3955-3960 (2014)) in a microfluidic device (Chen, D., et al., J Am Chem Soc, 134:6948-6951 (2012)). This LNP delivers oligonucleotides to hepatocytes in vivo (Yin, H., et al., Nat Biotechnol, (2017); (Dong, Y., et al., PNAS, 111:3955-3960 (2014)). Hepatocyte-targeting LNPs were formulated to carry iOligo, or as a control, the scrambled sequence. Hepatocyte-targeting LNPs to carry chemically modified sgGFP were also formulated. In all three cases, small, stable LNPs with low polydispersity were formed. Mice that express SpCas9-GFP under a ubiquitous CAG promoter (Platt, R. J., et al., Cell, 159:440-445 (2014)) were injected with either iOligo or the control oligo, and two hours later, the same mice were injected with sgGFP (FIG. 4B). Five days later, we sacrificed the mice, isolated hepatocytes (CD31-CD45-) using fluorescence activated cell sorting (FACS), and quantified GFP protein expression as well as indels.

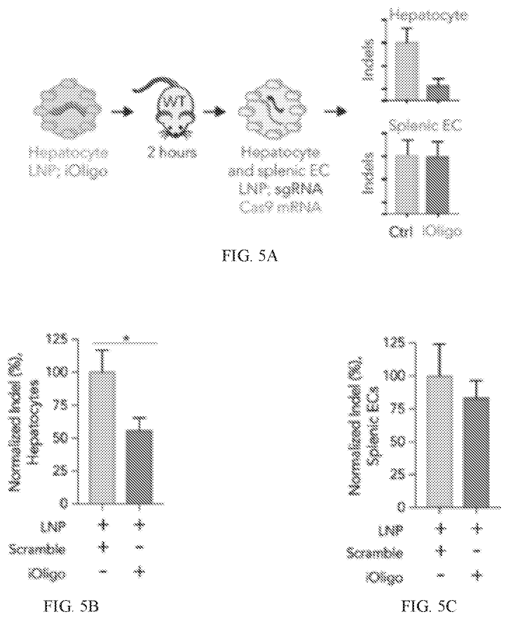

iOligos were then tested in wild type C57BL/6 mice, a model that is more clinically relevant than transgenic mice expressing Cas9. The iOligo or scramble control were formulated into the hepatocyte-targeting LNP, then administered intravenously to wild type adult mice (FIG. 5A). Two hours later, the mice were injected with LNPs carrying Cas9 mRNA and a chemically modified sgRNA targeting ICAM-2 (Platt, et al., Cell, 159:440-445 (2014)). Wild type mice were not injected with sgGFP since they did not have GFP in their genome. Importantly, for the second injection, LNPs that deliver Cas9 mRNA and sgRNA to splenic endothelial cells and hepatocytes were utilized (Sago, C., et al., PNAS, 115: E9944-E9952 (2018)).

### Results:

Compared to control mice injected with PBS, GFP expression in mice injected with control oligo and sgGFP was reduced by 50% as measured by mean fluorescent intensity (MFI) (FIG. 4C). GFP expression in mice treated with iOligo and sgGFP was statistically higher, suggesting that iOligo blocked sgGFP gene editing in Cas9 mice (FIG. 4C). Indel percentages decreased by 58% in iOligo treated mice, relative to mice treated with the control oligo (FIG. 4D), suggesting the effect was Cas9-mediated.

After isolating splenic endothelial cells (CD31+CD45−) and hepatocytes using FACS, we found that pre-delivery of iOligos to hepatocytes resulted in a statistically significant reduction in hepatocyte indels (FIG. 5B), but not splenic endothelial cell indels (FIG. 5C). In both in vivo experiments, iOligos were well tolerated by mice. These data led to the conclusion that iOligo delivery to hepatocytes can reduce hepatocyte editing without reducing editing in other cell types within the same animal.

### Example 3. Combining iOligo and siRNA Potently Reduces Gene Editing In Vivo

### Materials and Methods:

The combination of iOligo (which targets the sgRNA) and the siRNA approach (which targets Cas9 mRNA) on editing in vivo was then tested (FIG. 5C). Mice were intravenously injected with hepatocyte-targeting LNPs carrying siGFP, then 14 hours later, mice were injected with hepatocyte-targeting LNPs containing iOligo. Two hours later, mice were intravenously injected with Cas9 mRNA and sgRNA in LNPs that edit splenic cells and hepatocytes. To compare the combination of iOligo and siUTR, the control groups of iOligo paired with control siRNA, as well as scramble iOligo paired with siGFP were included.

### Results:

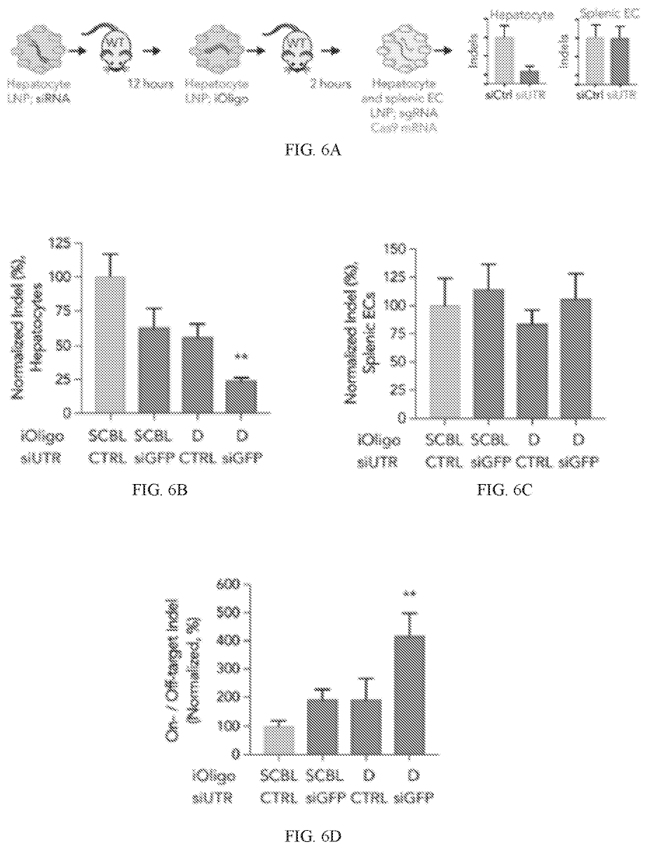

Combining iOligo and siGFP potently reduced editing in hepatocytes (FIG. 6B) and splenic editing was not reduced (FIG. 6C). This led to preferential editing in the spleen (FIG. 6D). The combinations of iOligo and siUTR were well tolerated in mice. These data are particularly exciting given the number of ways iOligo and siUTR combinations could be optimized.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A pharmaceutical composition comprising:
a plurality of nanoparticles comprising an effective amount of a genome editing antagonist oligonucleotide having a nucleic acid sequence complementary to at least a portion of a tracrRNA sequence of an sgRNA, wherein the oligonucleotide blocks, inhibits and/or interferes with the interaction of the sgRNA and an RNA-guided DNA endonuclease.

2. The pharmaceutical composition of claim 1, wherein the genome editing antagonist oligonucleotide hybridizes to at least a portion of the tracrRNA sequence of the sgRNA.

3. The pharmaceutical composition of claim 1, wherein the genome editing antagonist oligonucleotide is chemically modified to increase stability, reduce immunogenicity, and/or increase affinity between the genome editing antagonist oligonucleotide and the sgRNA.

4. The pharmaceutical composition of claim 3, wherein the modification is 2′O-Methyl ribose, phosphorothioate, or both.

5. The pharmaceutical composition of any one of claims 1-4, wherein the nanoparticles preferentially target hepatocytes.

6. The pharmaceutical composition of any one of claims 1-5, wherein the nanoparticles are lipid nanoparticles.

7. The pharmaceutical composition of claim 6, wherein the lipid nanoparticles comprise C₁₄PEG₂₀₀₀, cholesterol, 1,2-distearoyl-sn-glycero-3-phosphocholine (DSPC), and an ionizable lipid, wherein the ionizable lipid is cKK-E12.

8. The pharmaceutical composition of claim 6 or 7, wherein the lipid nanoparticles comprises about 30 mol % to about 80 mol cKK-E12, about 5 mol % to about 55 mol cholesterol, about 10 mol % to about 35 mol % phospholipid, and about 0 mol % to about 20 mol % PEG-lipid.

9. The pharmaceutical composition of any one of claims 1-8, wherein the genome editing antagonist oligonucleotide has a nucleic acid sequence that is 80% or more homologous, 85% or more homologous, 90% or more homologous, 95% or more homologous, and/or 100% homologous to any one of SEQ ID NOs:5-8.

10. A pharmaceutical composition comprising:
a plurality of first nanoparticles comprising a genome editing antagonist oligonucleotide having a first nucleic acid sequence complementary to at least a portion of a tracrRNA sequence of an sgRNA, wherein the oligonucleotide blocks, inhibits and/or interferes with the interaction of the sgRNA and an RNA-guided DNA endonuclease;
a plurality of second nanoparticles comprising a second nucleic acid sequence encoding the RNA-guided DNA endonuclease; and
a plurality of third nanoparticles comprising the sgRNA, wherein the sgRNA comprises a third nucleic acid sequence comprising a crRNA sequence having complementarity to a fourth nucleic acid sequence encoding a target gene fused to a fifth nucleic acid sequence comprising the tracrRNA sequence.

11. The pharmaceutical composition of claim 10, wherein the tracrRNA has a nucleic acid sequence that is 80% or more homologous, 85% or more homologous, 90% or more homologous, 95% or more homologous, and/or 100% homologous to SEQ ID NO:1.

12. The pharmaceutical composition of claim 10, wherein the genome editing antagonist oligonucleotide has a nucleic acid sequence is 80% or more homologous, 85% or more homologous, 90% or more homologous, 95% or more homologous, and/or 100% homologous to any one of SEQ ID NOs:5-8.

13. The pharmaceutical composition of any one of claims 10-12, wherein the genome editing antagonist oligonucleotide is chemically modified to increase stability, reduce immunogenicity, and/or increase affinity between the genome editing antagonist oligonucleotide and the sgRNA.

14. The pharmaceutical composition of claim 13, wherein the modification is 2′O-Methyl ribose, phosphorothioate, or both.

15. The pharmaceutical composition of any one of claims 10-14, wherein the first nanoparticles passively target hepatocytes.

16. The pharmaceutical composition of any one of claims 10-15, wherein the nanoparticles are lipid nanoparticles.

17. The pharmaceutical composition of claim 16, wherein the lipid nanoparticles comprise C₁₄PEG₂₀₀₀, cholesterol, 1,2-distearoyl-sn-glycero-3-phosphocholine (DSPC), and an ionizable lipid, wherein the ionizable lipid is cKK-E12.

18. The pharmaceutical composition of claim 16 or 17, wherein the lipid nanoparticles comprise about 30 mol % to about 80 mol % cKK-E12, about 5 mol % to about 55 mol % cholesterol, about 10 mol % to about 35 mol phospholipid, and about 0 mol % to about 20 mol % PEG-lipid.

19. The pharmaceutical composition of any one of claims 10-18, wherein the RNA guided DNA endonuclease is selected from the group consisting of Cas9, CasX, CasY, Cas13, and Cpf1.

20. The pharmaceutical composition of any one of claims 10-19, wherein the second nanoparticles and the third nanoparticles are formulated to deliver nucleic acids to splenic endothelial cells and/or lung endothelial cells.

21. The pharmaceutical composition of claim 20, wherein one or both of the second and third nanoparticles comprise 7C1:cholesterol:C₁₄-PEG₂₀₀₀:18:1 lyso PC at a molar ratio of 50:23.5:6.5:20 or 7C1:cholesterol:C₁₄-PEG₂₀₀₀:DOPE at a molar ratio of 60:10:25:5.

22. A method of inhibiting RNA-guided gene editing in hepatocytes in a subject in need thereof comprising:
pre-treating the subject with an effective amount of a pharmaceutical composition comprising a genome editing antagonist oligonucleotide having a nucleic acid sequence complementary to at least a portion of a tracrRNA sequence of an sgRNA, wherein the oligonucleotide blocks, inhibits and/or interferes with the interaction of the sgRNA and an RNA-guided DNA endonuclease, and wherein the pharmaceutical composition is formulated to deliver to hepatocytes, and
after a period of time systemically administering to the subject a RNA-guided genome editing system in an amount effective to perform genome editing in cells,
wherein the effective amount of the pharmaceutical composition inhibits the activity of the RNA-guided genome editing system in hepatocytes.

23. The method of claim 22, wherein the RNA-guided genome editing system comprises an RNA-guided endonuclease and an sgRNA.

24. The method of claim 22, wherein the RNA-guided DNA endonuclease is Cas9.

25. The method of claim 22, wherein the genome editing antagonist oligonucleotide is delivered in a nanoparticle.

26. The method of claim 22, wherein the RNA-guided genome editing system is administered systemically.

27. A method of treating a genetic disease or disorder in a subject in need thereof comprising,
pre-treating the subject with an effective amount of a pharmaceutical composition comprising a genome editing antagonist oligonucleotide having a nucleic acid sequence complementary to at least a portion of a tracrRNA sequence of an sgRNA, wherein the oligonucleotide blocks, inhibits and/or interferes with the interaction of the sgRNA and an RNA-guided DNA endonuclease, and wherein the pharmaceutical composition is formulated to deliver to hepatocytes, and
after a period of time administering to the subject an RNA-guided genome editing system in an amount effective to perform RNA-guided genome editing in diseased cells,
wherein the effective amount of the pharmaceutical composition inhibits the activity of the RNA-guided genome editing system in hepatocytes and genome editing occurs in other cell types, including the diseased cells.

28. The method of claim 27, wherein the RNA-guided genome editing system is administered systemically.

29. The method of claim 27, wherein the genome editing antagonist oligonucleotide is administered to the subject 1, 2, 3, 4, or 5 hours before the RNA-guided genome editing system.

30. A kit comprising:
a plurality of first nanoparticles comprising a genome editing antagonist oligonucleotide having a first nucleic acid sequence complementary to at least a portion of a tracrRNA sequence of an sgRNA, wherein the oligonucleotide blocks, inhibits and/or interferes with the interaction of the sgRNA and an RNA-guided DNA endonuclease;
a plurality of second nanoparticles comprising a second nucleic acid sequence encoding the RNA-guided DNA endonuclease; and
a plurality of third nanoparticles comprising the sgRNA, wherein the sgRNA comprises a third nucleic acid sequence comprising a crRNA sequence having complementarity to a fourth nucleic acid sequence encoding a target gene fused to a fifth nucleic acid sequence comprising the tracrRNA sequence.
